(19) United States
(12) Patent Application Publication
(10) Pub. No.: US 2026/0167916 A1
(43) Pub. Date: Jun. 18, 2026

(54) CONTINUOUS-FLOW MAGNETIC FIELD-ASSISTED FERMENTATION AND CATALYTIC SYNTHESIS DEVICE AND METHOD

(71) Applicant: BIOMAG INNOVATIONS LLC, SEBASTIAN, FL (US)

(72) Inventors: Na YANG, SEBASTIAN, FL (US); Yuyi ZHOU, SEBASTIAN, FL (US); Zhiyuan XU, SEBASTIAN, FL (US); Yamei JIN, SEBASTIAN, FL (US); Rongming LI, SEBASTIAN, FL (US)

(73) Assignee: BIOMAG INNOVATIONS LLC, SEBASTIAN, FL (US)

(21) Appl. No.: 19/084,824

(22) Filed: Mar. 20, 2025

(30) Foreign Application Priority Data
Dec. 16, 2024 (CN) 2024118509664

(51) Int. Cl. C12M 1/42; C12M 1/00; C12M 1/40

(52) U.S. Cl. CPC C12M 35/02; C12M 21/18; C12M 29/06

(57) ABSTRACT

Provided are a continuous-flow magnetic field-assisted fermentation and catalytic synthesis device and method. The device includes a heat-insulating container, a magnetic field assembly, and a sample tube cavity. The magnetic field assembly is fixed inside the heat-insulating container; the heat-insulating container is of a closed structure with an inlet and an outlet, and a constant-temperature medium can be introduced and circulated in the heat-insulating container. The sample tube cavity penetrates through the magnetic field assembly, and a sample tube can all pass through the sample tube cavity. Fermentation liquid or a reaction medium continuously flows in the sample tube and is subjected to the magnetic field for the treatment; and an included angle between a direction of a magnetic field line of the magnetic field and a flowing direction of the fermentation liquid or reaction medium ranges from 0° to 90°.

### CROSS-REFERENCE TO THE RELATED APPLICATIONS

This application is based upon and claims priority to Chinese Patent Application No. 202411850966.4, filed on Dec. 16, 2024, the entire contents of which are incorporated herein by reference.

### TECHNICAL FIELD

The present disclosure particularly relates to a continuous-flow magnetic field-assisted fermentation and catalytic synthesis device and method, belonging to the technical field of biological fermentation and catalytic synthesis.

### BACKGROUND

Because of limited environmental resources, it is necessary to adopt a low-carbon and sustainable development strategy to reduce environmental pressure caused by the energy demand. Novel fermentation culture and chemical synthesis technologies provide an effective way to obtain high-quality and high-yield products, such as bioactive substances, fuels, foods, fine chemicals, and pharmaceuticals. In recent years, with the in-depth research of the non-thermal effect caused by a magnetic field, magnetic field-assisted fermentation, cell culture and chemical synthesis have become popular. It is found in the related research that the magnetic field can regulate and control the process of mass transfer, signal transmission, cell metabolism and catalytic reaction through macroscopic and microscopic ways. The principle includes three aspects: (1) biological intracellular substances such as proteins, nucleic acids, enzymes and free radicals are affected by the magnetic field, that is, Lorentz force effect, eddying effect, induced current effect and an additional magnetic moment, to induce metabolic activities and changes in physicochemical characteristics; (2) the effect of magnetic field on water cluster and its polarization as well as on ion migration promotes the release of dissolved oxygen or carbon monoxide inside; and (3) the effect of magnetic field on cell membrane changes its permeability and flowability, thus regulating the process of ion transmembrane transport, signal transduction and electron transfer in cells. Based on the above research, appropriate magnetic field condition is conducive to promoting biotransformation rate and catalytic reaction rate for the fermentation and synthesis reactions.

In the prior art, the existed magnetic field devices mostly adopt batch treatment for the fermentation and other reactions, a permanent magnet or a metal coil is arranged outside a fermentation tank or a reactor, making the feed liquid or sample medium subjected to the magnetic field, which undoubtedly increases the complicacy of the fermentation tank or reaction system. Meanwhile, the controllability of the magnetic field strength and degree of homogeneity is insufficient, and the range of operation parameters is limited, i.e., low magnetic field energy caused by low frequency as well as low degree of homogeneity, leading to a poor effect on biological fermentation culture and synthetic catalytic reaction. Therefore, continuous-flow magnetic field-assisted fermentation and catalysis devices have attracted much attention. However, the traditional magnetic field assembly cannot ensure that samples are subjected to consistent magnetic flux during the process, that is, the uneven distribution of magnetic field will cause that there is no repeatability on application effect. Then the action of applied magnetic field on the biological fermentation or catalytic synthesis reaction cannot be effectively implemented. Therefore, the distance between feed liquid and the magnetic field assembly as well as a relationship between a flowing direction and a direction of the magnetic field lines are critical parameters to avoid the generation of inhomogeneity magnetic field environment and thus ensure a stable magnetic flux. In addition, if extra excitation energy for the generation of the magnetic field cannot be dissipated, the heat loss will be transferred to fermentation liquid or a reaction medium, increasing the temperature of the system or generating temperature fluctuation, which disturbs the fermentation or reaction process. In this case, the application effect is unstable, and a magnetic field with high frequency in large space fermentation and reaction cannot be achieved, making the large-scale application of the method limited.

### SUMMARY

A main objective of the present disclosure is to provide a continuous-flow magnetic field-assisted fermentation and catalytic synthesis device and method, so as to overcome the deficiencies in the prior art.

To achieve the above objective, the present disclosure adopts the following technical solutions:

A first aspect of embodiments of the present disclosure provides a continuous-flow magnetic field-assisted fermentation and catalytic synthesis device, including:
a constant-temperature unit, including a heat-insulating container, and a circulating constant-temperature medium supply system. The heat-insulating container has a constant-temperature cavity and x sample tube cavities penetrating therethrough, the sample tube cavities are encapsulated by the constant-temperature cavity and in heat-conducting fit with the constant-temperature cavity, and the circulating constant-temperature medium supply system is connected to the constant-temperature cavity to form a fluid loop for continuous-flow of the constant-temperature medium;
a reaction generation unit, including a sample container, and y sample tubes, where the sample container is at least configured to accommodate sample medium and provide an environment required for fermentation culture or catalytic synthesis reaction of the sample medium, each of the sample tube is connected to the sample container to form a fluid loop for the sample medium to flow continuously, and at least a part of each of the sample tubes is arranged in one sample tube cavity and is in heat-conducting fit with the sample tube cavity; and
a magnetic field generation unit, including y magnetic field assemblies, where the y magnetic field assemblies are arranged in the constant-temperature cavity, each of the magnetic field assemblies is correspondingly distributed around one sample tube cavity, the magnetic field assembly is at least configured to generate a magnetic field inside the sample tube cavity. An included angle between a direction of the magnetic field line generated by the magnetic field assembly and either an axial direction of the sample tube passing through each of the magnetic field assemblies or a flowing direction of the sample medium within the sample tube ranges from 0° to 90°, the magnetic field can change physicochemical parameters or biochemical indicators of sample medium in the sample tube, x≥y≥1, and x and y are positive integers.

In a specific embodiment, the constant-temperature unit includes y heat-dissipating structures. The y heat-dissipating structures are arranged in the constant-temperature cavity, and each of the heat-dissipating structures is arranged around one sample tube cavity and is in heat-conducting fit with the sample tube cavity.

Further, the constant-temperature cavity includes one first constant-temperature cavity, and y second constant-temperature cavities. The y second constant-temperature cavities are surrounded by and connected to the first constant-temperature cavity. Each second constant-temperature cavity is arranged around one sample tube cavity and is in heat-conducting fit with the sample tube cavity. The second constant-temperature cavities and constant-temperature mediums filled in the second constant-temperature cavities serve as the heat-dissipating structures.

Further, the heat-insulating container includes a first housing, and x second housings. The first housing is fixedly combined with the x second housings, the first housing and the x second housings are enclosed to form the constant-temperature cavity, and each of the second housing is enclosed to form the sample tube cavity. The first housing is made of a metallic material, or a nonmetallic material, and the second housing is made of a nonmetallic material. Specifically, the metallic material includes aluminum, iron, or stainless steel, etc., and the nonmetallic material includes polytetrafluoroethylene (PTFE), polyether ether ketone (PEEK), bakelite, glass, perfluoroalkoxy (PFA), polyethylene, or epoxy resin, etc.

Further, the magnetic field assembly is distributed in the heat-dissipating structure, or the magnetic field assembly is distributed around the sample tube cavity.

Further, the magnetic field assembly includes a metal coil, which is wound around the periphery of the sample tube cavity, and the metal coil is electrically connected to a power supply to generate the magnetic field; or the magnetic field assembly includes at least one permanent magnet.

Further, a wire diameter of the metal coil ranges from 0.01 mm to 5 mm.

Further, the number of turns of each metal coil ranges from 10 to 30000.

Further, the magnetic field is an alternating magnetic field, or a static magnetic field. The alternating magnetic field is generated by introducing alternating current into the metal coil, and the static magnetic field is generated by introducing direct current into the metal coil; or the static magnetic field is generated by the permanent magnet.

Further, the metal coil is electrically connected to a power supply, and a current provided by the power supply ranges from 0.1 A to 16 A. The power supply is a direct-current power supply, or an alternating-current power supply.

Further, the y magnetic field assemblies are arranged in parallel, and a ratio of an outer perimeter of the magnetic field assembly to the center-to-center distance between the two adjacent magnetic field assemblies ranges from (1:0.15) to (1:20), e.g., 1:0.15, 1:0.3, 1:1.2, 1:2.6, 1:4.3, 1:5.5, 1:6, 1:7.5, 1:9, 1:10, 1:15, 1:20 and the like.

Further, a part, located within the sample tube cavity, of the sample tube is a straight tube, or spiral tube.

Further, the constant-temperature unit includes a temperature regulator, and a temperature sensor. The temperature regulator is connected to the heat-insulating container and the circulating constant-temperature medium supply system. The temperature regulator, the heat-insulating container and the circulating constant-temperature medium supply system form a fluid loop for continuous-flow of the constant-temperature medium. The temperature regulator is at least configured to maintain a temperature of the constant-temperature medium in the fluid loop, and the temperature sensor is at least configured to monitor a temperature of the constant-temperature medium flowing into and/or out of the heat-insulating container.

Further, the constant-temperature medium may be water, mineral oil, or other cooling medium such as ethanol and propylene glycol.

A second aspect of embodiments of the present disclosure provides a continuous-flow magnetic field-assisted fermentation and catalytic synthesis method, including:
providing the continuous-flow magnetic field-assisted fermentation and catalytic synthesis device;
enabling sample medium in a sample container to pass through a sample tube in continuous-flow state;
enabling a magnetic field assembly to generate a magnetic field inside the sample tube, wherein an included angle θ between a direction of the magnetic field line generated by the magnetic field assembly and either an axial direction of the sample tube passing through each of the magnetic field assemblies or a flowing direction of the sample medium in the sample tube ranges from 0° to 90°, for example, θ may be 0°, 10°, 15°, 20°, 23°, 32°, 45°, 60°, 75°, 90° and the like; and
enabling a constant-temperature unit to maintain temperatures of the magnetic field assembly and the sample medium in the sample tube.

Further, the magnetic field is an alternating magnetic field or a static magnetic field, the alternating magnetic field has a magnetic field strength ranging from 0.1 mT to 500 mT and a frequency ranging from 0.1 Hz to 100 kHz. The static magnetic field has a magnetic field strength ranging from 0.1 mT to 600 mT.

Further, the flow rate of the sample medium in the sample tubes is 5 mL/h to 1000 L/h.

Further, the temperatures of the magnetic field assembly and the constant-temperature medium are maintained at −40° C. to 150° C., for example, may be −40° C., −30° C., −22° C., −10° C., 0° C., 15° C., 25° C., 55° C., 80° C., 100° C., 150° C., etc.

Further, the constant-temperature medium and the sample medium are delivered by a pump to flow, and the pump may be a centrifugal pump, a constant-flux pump, a screw pump, or a peristaltic pump, etc.

Compared with the prior art, the present disclosure has the following advantages:

The continuous-flow magnetic field-assisted fermentation and catalytic synthesis device provided by the present disclosure can implement the magnetic field-assisted treatment of the fermentation medium or reaction medium in a continuous-flow state. A distance between the medium in the sample tube and the magnetic field assembly as well as a relationship between the flowing direction and the direction of the magnetic field line are limited to make the sample medium or feed liquid subject to a homogeneous and stable magnetic field. The experiment shows that this device has reliable repeatability, since the non-thermal effect on the biological fermentation or catalytic synthesis reactions can be achieved by regulating an appropriate magnetic field environment. In addition, an intermediate-frequency magnetic field with high energy density can be generated by the magnetic field assembly with this heat-dissipating structure, so the large-scale application of the technology can be achieved.

According to the present disclosure, the efficient and stable process of the fermentation and chemical catalysis exposed to the magnetic field is implemented. In addition, the included angle between the direction of the magnetic field line of the magnetic field and the flowing direction of the sample medium ranges from 0° to 90°. For charged solutes and free ions in the fermentation solution or reaction medium, since there is a certain included angle between the flowing direction of the sample medium and the direction of the magnetic field line, the strength and direction of the Lorentz force on charged solutes and free ions in the fluid will show a specific regulation effect on the yield and product quality. Furthermore, under the action of stable magnetic flux, the ion exchange and substance transfer in cell membrane channel during the fermentation or chemical reaction can be promoted. That is, when the distribution chart of angles of electron cloud as well as the orientation action of diamagnetic component are influenced, the change of their induced magnetic field will be caused. And the direction and strength of the induced magnetic field force are related to a migration state of the solutes or ions. Moreover, the induced magnetic fields in different directions also belong to vectors, the interaction of two vectors will further produce a synergistic effect on the sample medium to influence the fermentation or reaction process, such as reaction potential energy and the diffusion of the solutes, thereby improving the growth efficiency, metabolic yield or reaction rate, etc.

### BRIEF DESCRIPTION OF THE DRAWINGS

FIG. 1 is a schematic structural diagram of a continuous-flow magnetic field-assisted fermentation and catalytic synthesis device according to a typical embodiment of the present disclosure;

FIG. 2A and FIG. 2B are partial schematic structural diagrams of a continuous-flow magnetic field-assisted fermentation and catalytic synthesis device according to Embodiment 1 of the present disclosure;

FIG. 3A and FIG. 3B are partial schematic structural diagrams of a continuous-flow magnetic field-assisted fermentation and catalytic synthesis device according to Embodiment 2 of the present disclosure;

FIG. 4A and FIG. 4B are partial schematic structural diagrams of a continuous-flow magnetic field-assisted fermentation and catalytic synthesis device according to Embodiment 3 of the present disclosure.

### DETAILED DESCRIPTION OF THE EMBODIMENTS

In view of the shortcomings in the prior art, the inventors have been able to put forward the technical solution of the present disclosure through long-term research and a lot of practice. The technical solutions and the implementation process and principle thereof are further explained and described below with reference to the accompanying drawings and the specific embodiments. It should be noted that a controller, a power supply, a refrigeration compressor, a heater and a temperature sensor used in the present disclosure are all known to those skilled in the art and can all be obtained from the market. The above technical features of the present disclosure and the technical features described in detail in the following (embodiments) can be combined with each other to form a new or preferred technical solution, which will not elaborated here due to limited space.

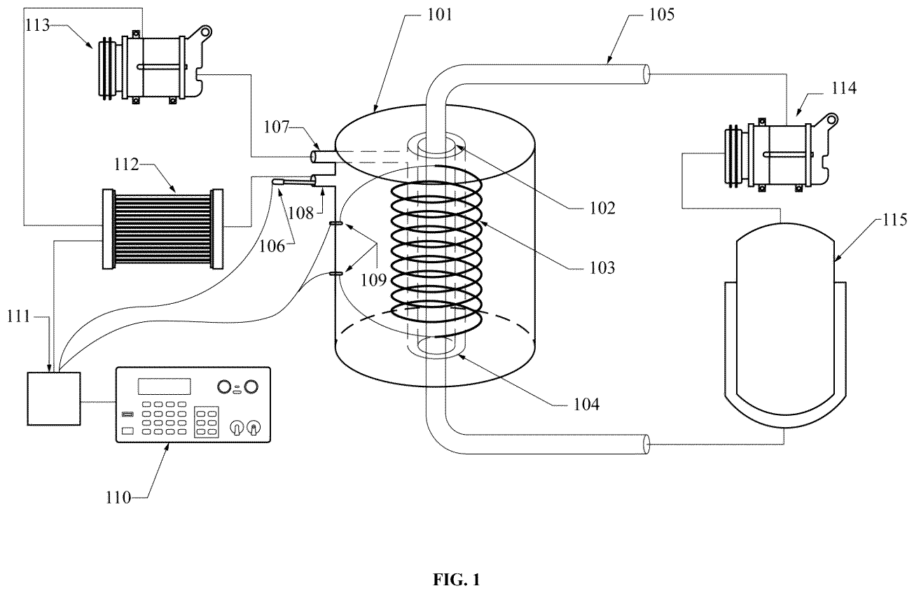

In a specific embodiment, referring to FIG. 1, a continuous-flow magnetic field-assisted fermentation and catalytic synthesis device includes: a heat-insulating container 101, x magnetic field assemblies 103, x sample tubes 105, a power supply 110, a controller 111, a temperature regulator 112, a constant-temperature medium pump 113, a sample pump 114 and a sample container 115.

The heat-insulating container 101 includes a first housing, and x second housings. The first housing and the x second housings are fixedly combined, the first housing and the x second housings are enclosed to form a closed constant-temperature cavity, and each of the second housings is enclosed to form a sample tube cavity 102 connected to the first housing. The constant-temperature cavity includes a first constant-temperature cavity, and x second constant-temperature cavities 104. The x second constant-temperature cavities 104 are surrounded and connected the first constant-temperature cavity, and each of the second constant-temperature cavities 104 is arranged around the periphery of one sample tube cavity 102 and in heat-conducting fit with the sample tube cavity 102 through the second housing. The first housing is provided with a constant-temperature medium inlet 107, and a constant-temperature medium outlet 108. A temperature sensor 106 is arranged in the constant-temperature outlet 108 or/and constant-temperature medium inlet 107, the constant-temperature medium pump 113 is connected to the temperature regulator 112, the constant-temperature medium pump 113 and the temperature regulator 112 are further connected to the constant-temperature medium inlet 107 and the constant-temperature medium outlet 108 respectively through conduits. The constant-temperature medium pump 113, the temperature regulator 112 and the constant-temperature cavity form a fluid loop for the continuous-flow of constant-temperature medium.

The temperature regulator 112 and the temperature sensor 106 are connected to the controller 111, and a temperature parameter of the constant-temperature medium can be regulated and monitored through the controller 111.

The x magnetic field assemblies 103 are arranged inside the constant-temperature cavity, and each of the magnetic field assemblies 103 surrounds one sample tube cavity 102 and is configured to generate a magnetic field.

A part of each sample tube 105 is arranged in the sample tube cavity 102, and both ends of the sample tube 105 are connected to the sample pump 114 and the sample container 115 through conduits, respectively. The sample pump 114 is further connected to the sample container 115, the sample tube 105, the sample pump 114 and the sampler container 115 form a liquid loop for the continuous-flow of sample medium. The ion exchange and the inter-substance interaction in the medium flowing through the sample tube 105 located in the sample tube cavity 102 can be changed by the applied magnetic field.

Specifically, the constant-temperature medium can be delivered by the constant-temperature medium pump 113 into the constant-temperature cavity for continuous flow. The magnetic field assembly 103 is immersed into the constant-temperature medium, and the operation temperatures of the magnetic field assembly 103 can be maintained between −40° C. and 150° C. by using the temperature regulator 112 and the controller 111. The constant-temperature mediums can fill the first constant-temperature cavity and the second constant-temperature cavity 104, and a temperature in the sample tube cavity 102 is thus regulated by the constant-temperature medium in the second constant-temperature cavity 104 and the second housing. Specifically, the temperature of the constant-temperature medium is determined by the temperature sensor 106. Specifically, the temperature regulator 112 may be a refrigeration compressor, a heat exchanger or a circulating air cooler; the constant-temperature medium pump 113 may be a centrifugal pump, a constant-flux pump, a screw pump, or a peristaltic pump; the temperature senor 106 may be a PT100 thermal resistor, a K type thermocouple, or an infrared thermal imaging system; and the constant-temperature medium may be water, mineral oil, or the other refrigerant. It should be noted that on one hand, the constant-temperature medium can perform heat dissipation and temperature control on the magnetic field assembly 103, and on the other hand, the constant-temperature medium can regulate the temperature of the sample tube cavity 102 to maintain the temperature of the sample medium through heat conduction.

Specifically, the heat-insulating container 101 has a closed structure provided with a constant-temperature medium inlet 107 and a constant-temperature medium outlet 108. Specifically, the first housing and the second housing can be fixed and closed by means of welding, gluing, fastener connection and other processes. The first housing may be made of a metallic material, or a nonmetallic material. The sample tube cavity 102 has machinability. The second housing is made of a nonmetallic material with a certain mechanical strength. The radial section of the sample cavity 102 may be circular, rectangular, or other polygons. Exemplarily, the metallic material may be aluminum, iron or stainless steel, etc., and the nonmetallic material includes polytetrafluoroethylene (PTFE), polyether-ether-ketone (PEEK), bakelite, glass, perfluoroalkoxy (PFA), polyethylene, or epoxy resin, etc.

Specifically, the magnetic field assembly may be arranged in the first constant-temperature cavity and distributed around the periphery of the second constant-temperature cavity 104, or the magnetic field assembly is arranged in the second constant-temperature cavity 104 and arranged between the sample tube cavities 102 in a surrounding mode. Specifically, an included angle between a direction of a magnetic field line of a magnetic field generated by the magnetic field assembly 103 and an axial direction of the sample tube 105 passing through the magnetic field assembly 103 ranges from 0° to 90°.

Specifically, the magnetic field assembly 103 may be a metal coil, or at least one permanent magnet. Specifically, when the magnetic field assembly 103 is the metal coil, the metal coil may be totally or partially located around the periphery of the second constant-temperature cavity 104 or the sample tube cavity 102. A binding post 109 is arranged on the heat-insulating container 101, the metal coil is electrically connected to the controller 111 and the power supply 110 through the binding post 109, and the current provided by the power supply 110 causes the metal coil to generate the magnetic field. Specifically, the wire diameter of the metal coil ranges from 0.01 mm to 5 mm, the number of turns of the metal coil ranges from 10 to 30000. Exemplarily, the metal coil may be Helmholtz coil, a solenoid coil, and the like. When the magnetic field assemblies 103 are one or more pairs of permanent magnets, the one or more pairs of permanent magnets are fixed around the periphery of the second constant-temperature cavity 104 or the sample tube cavity 102.

Specifically, the magnetic field assembly 103 may generate an alternating magnetic field with a magnetic field strength ranging from 0.1 mT to 500 mT and a frequency ranging from 0.1 Hz to 100 kHz, or generate a static magnetic field with a magnetic field strength ranging from 0.1 mT to 600 mT. The alternating magnetic field is generated by the alternating current provided by, the power supply 110. The static magnetic field is generated by the direct current provided by the power supply 110, or the static magnetic field is generated by the permanent magnet. Furthermore, the power supply 110 is connected to the controller 111, and power supply parameters and magnetic field parameters provided by the magnetic field assembly can be regulated by the controller 111.

Specifically, the x magnetic field assemblies 103 should be fixed inside the heat-insulating container 101. When x≥2, the x magnetic field assemblies 103 should be arranged in parallel; furthermore, a ratio of an outer perimeter of each of the magnetic field assemblies 103 to the center-to-center distance between the two adjacent magnetic field assemblies 103 should range from (1:0.15) to (1:20). Through this design, the x magnetic field assemblies 103 may perform fermentation or catalytic synthesis treatment to increase the volumetric capacity, and parameters of the magnetic field environments in the x sample tubes 105 can be ensured to be basically the same. If the adjacent magnetic field assemblies 103 are too far away from each other, there is excessive volume of the whole device, and the magnetic fields between the adjacent magnetic field assemblies 103 are isolated from each other. If the adjacent magnetic field assemblies 103 are too close to each other, the mutual heat dissipation between the magnetic field assemblies will be disturbed. Meanwhile, the x magnetic field assemblies 103 are arranged in parallel, the x magnetic field assemblies 103 have the same direction and distribution of magnetic field lines, there is an enhancement effect on the application among the magnetic fields produced by the x magnetic field assemblies 103, otherwise, the effect of the produced magnetic fields will weaken because of their different angles or opposite directions.

It should be noted that the outer perimeter of the magnetic field assembly 103 refers to a length of a circumferential profile of the magnetic field assembly 103, which may specifically be understood as a perimeter of an orthogonal projection pattern formed by the magnetic field assembly 103 in an axial direction thereof. Exemplarily, when the magnetic field assembly 103 refers to the metal coil, the outer perimeter may be a length of a single winding of annular coil. When the magnetic field assembly 103 refers to a single annular permanent magnet, the outer perimeter is a circumferential length of an outer ring of an end face of the annular permanent magnet. When the magnetic field assembly 103 refers to multiple permanent magnets, the multiple permanent magnets are distributed in an annular area, and the outer perimeter is the circumferential length of an outer ring of the annular area. The center-to-center distance between the two adjacent magnetic field assemblies 103 is a vertical distance between the geometric centers of the two adjacent magnetic field assemblies 103. When the two magnetic field assemblies 103 present a regular geometric structure as a whole, such as a cylinder structure or a prism structure, the center-to-center distance between the two adjacent magnetic field assemblies 103 may also be regarded as a linear distance between the geometric centers of two orthogonal projection patterns formed by the two adjacent magnetic field assemblies 103 in the axial directions thereof.

Specifically, the sample tube cavity 102 penetrates through the heat-insulating container 101, and the sample tube 105 for the continuous-flow of sample medium (fermentation liquid or a synthetic reaction mixture) can pass through the sample tube cavity 102. The sample medium can flow continuously inside the sample tube 105 by the sample pump 114; and an included angle θ between the direction of the magnetic field line generated by the magnetic field assembly 103 and the flowing direction of the sample medium in the sample tube 105 ranges from 0° to 90°. Exemplarily, the sample tubes 105 all pass through the sample tube cavity 102, and sample medium needing to be treated that is delivered into the sample tube 105 by the sample pump 114 to flow continuously. In addition, the sample medium can perform fermentation or catalytic reaction in an external sample container 115. Exemplarily, the sample container 115 may be a fermentation tank, or a reaction kettle. It should be noted that a part, located in the sample tube cavity 102, of the sample tube 105 is a straight tube, or a spiral tube, the difference between the straight tube and the spiral tube is that an interaction angel of the magnetic field line and the sample flowing direction is different. When the sample tube 105 is the spiral tube, the included angle θ between the direction of the magnetic field line generated by the magnetic field assembly 103 and the flowing direction of the sample medium in the sample tube 105 is closer to 90°. Furthermore, under the same magnetic field line distribution, due to the longer length of the spiral tube, the duration of the process is thus longer, and the fermentation or catalytic reaction is more sufficient.

Specifically, the controller 111 may be a Programmable Logic Controller (PLC), or an integrated circuit board, or the like. The numerical control programs used in the controller can be readily obtained through commercial channels, and the circuit structures in the present disclosure are all implemented by methods or technologies known to those skilled in the art, which are not particularly limited here.

Specifically, the continuous-flow magnetic field-assisted fermentation and catalytic synthesis device can be applied in the fields of food, biology, life sciences, environment, materials, chemical industry, production of pharmaceuticals, etc.

### Embodiment 1

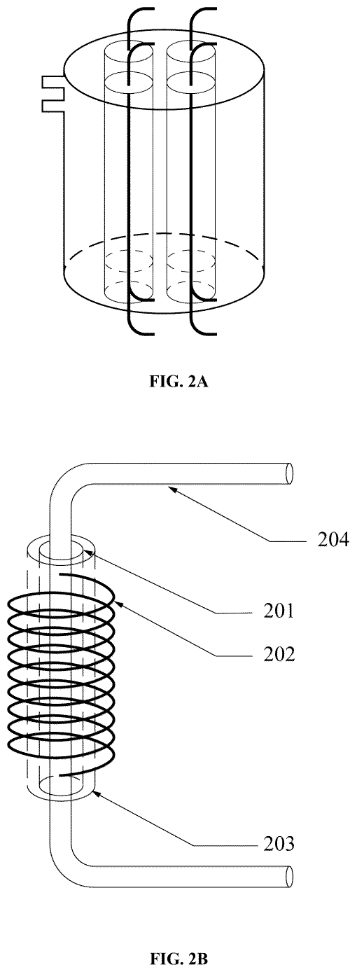

This embodiment provides a method for continuous-flow fermentation of Antrodia camphorate under a magnetic field, a device for fermenting Antrodia camphorate in this embodiment is a continuous-flow magnetic field-assisted fermentation and catalytic synthesis device in Embodiment 1, with the partial structures shown in FIG. 2A and FIG. 2B.

The continuous-flow magnetic field-assisted fermentation and catalytic synthesis device used in this embodiment is provided with four magnetic field assemblies 202, the magnetic field assemblies 202 are solenoid coils, where the wire diameter of the used solenoid coil is 2.0 mm, the number of turns of a single solenoid coil is 600 turns. The four magnetic field assemblies 202 are arranged in parallel and vertically fixed inside the heat-insulating container, respectively. A ratio of an outer perimeter of the solenoid coil to the center-to-center distance between the adjacent magnetic field assemblies 202 is 1:0.5. The sample tube cavity 201 passes through the heat-insulating container, it is made of a PFA (that is, the second housing is made of the PFA), and the solenoid coils are all wound around the periphery of the second constant-temperature cavity 203. The sample tubes 204 pass through the sample tube cavities 201, fermentation liquid is pumped to flow continuously in the sample tubes 204, and an included angle between the direction of the magnetic field line of the magnetic field and the flowing direction of the fermentation liquid in the sample tubes 204 is 0°. A constant-temperature mineral oil is pumped into the heat-insulating container, a refrigeration compressor or heat exchanger is configured to control the temperature of the constant-temperature mineral oil. A centrifugal pump is configured to circulate the constant-temperature mineral oil through the first and second constant-temperature cavities of the heat-insulating container, as well as inside and outside the magnetic field assembly to ensure uniform thermal distribution.

A method for continuous-flow fermentation of Antrodia camphorate under a magnetic field specifically includes:
(1) a preserved strain of Antrodia camphorata was inoculated onto a potato dextrose agar (PDA) slant medium and incubated at the constant temperature of 25° C. for two weeks to activate the strain;
(2) 1 cm² activated slant culture in Step (1) was transferred and incubated in 100 mL of seed culture medium at the constant temperature of 25° C. for one week to prepare a seed solution; and
(3) the Antrodia camphorata seed solution in Step (2) was inoculated into a liquid culture medium with an inoculation amount of 8% to serve as fermentation liquid, the fermentation liquid was pumped into a continuous-flow magnetic field-assisted fermentation and catalytic synthesis device, device parameters were set, 6 A excitation current was applied to a solenoid coil within the magnetic field assemblies 202 to generate an alternating magnetic field with a magnetic field strength of 20 mT and a frequency of 500 Hz, a fermentation temperature of the fermentation liquid was set to 25° C., a temperature of constant-temperature mineral oil was controlled by a refrigeration compressor and a heat exchanger, the centrifugal pump enabled constant-temperature mineral oil to flow in and out of the heat-insulating container circularly, a temperature of the sample tube cavity 201 was kept at 25° C., and the fermentation liquid was subjected to the alternating magnetic field for continuous-flow of constant-temperature culture at the flow rate of 5 L/h and at 25° C. for 5 days in the sample tube.

After that, the growth situation of Antrodia camphorata was observed, and the biomass and biomass yield increase rate of Antrodia camphorata fermentation liquid were calculated.

The specific calculation method is as follows:
biomass: 100 mL of the fermentation liquid was taken and centrifuged at 6000 g for 8 min, mycelium was collected, washed with distilled water for three times and dried in a 50° C. oven to a constant weight, and then the biomass (mg/mL) of Antrodia camphorata fermentation liquid per unit volume is obtained according to formula (2):

$$Fb = \frac{M}{V}$$

where Fb is biomass of Antrodia camphorata fermentation liquid per unit volume (mg/mL); M is mass mg of the mycelium after being dried to constant weight (mg); and V is the volume of the fermentation liquid (mL).

The result shows that in this embodiment, the biomass of the Antrodia camphorata fermentation liquid per unit volume reaches 0.742 mg/mL.

### Comparative Example 1

This example provides a method for fermenting Antrodia camphorata, the difference between Comparative Example 1 and Embodiment 1 is that the ratio of the outer perimeter of the solenoid coil to the center-to-center distance between the adjacent magnetic field assemblies is 1:0.14, that is, the solenoid coil is wound into a right triangle appearance, and the adjacent magnetic field assemblies are closely attached.

After that, the growth process of the Antrodia camphorata was observed, and the biomass of the Antrodia camphorata fermentation liquid was calculated. The result shows that the biomass of the Antrodia camphorata per unit volume in Comparative Example 1 is only 0.326 mg/mL.

By adopting the continuous-flow magnetic field-assisted fermentation and catalytic synthesis device provided by the present disclosure, compared with the close attachment of the magnetic field assemblies in Comparative Example 1, the biomass in Embodiment 1 is significantly increased by 127.61%. The fermentation liquid contains a large amount of carbon source, nitrogen source, inorganic salt, and water. These substances have diamagnetism, the reverse orientation of weak electron cloud magnetic moment occurs in a magnetic field environment, and a pore channel in cell membrane of the Antrodia camphorata tissue is adjusted to promote substance transfer, as well as the interaction of an active enzyme and a substrate, therefore, enhances the fermentation yield of the Antrodia camphorata and increases the biomass. In Comparative Example 1, the magnetic field assemblies are closely attached, the temperature of the magnetic field assembly is increased obviously, the temperature of the constant-temperature mineral oil and the sample tube cavity are thus increased sharply, and the sample in the sample tube has obvious temperature fluctuation. Meanwhile, the magnetic field assemblies are too close to each other, and the produced magnetic field lines interfere with each other, resulting in the inability to gather to generate a magnetic field with appropriate energy density, and further inhibition of the fermentation of the Antrodia camphorata.

### Embodiment 2

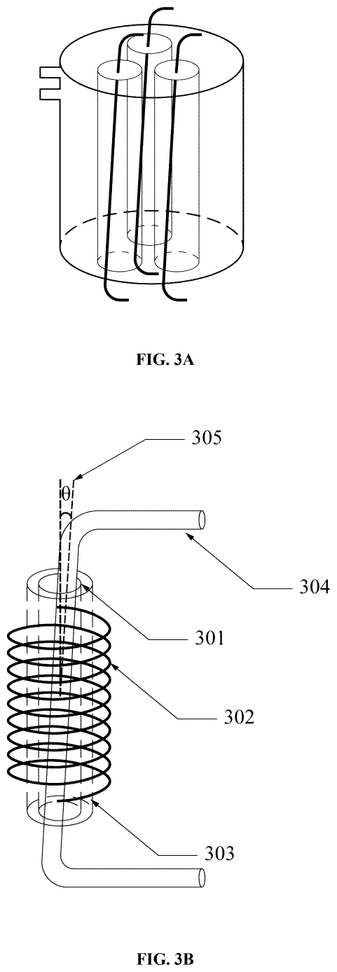

This embodiment provides a method for producing alcohol through continuous-flow magnetic field-assisted fermentation, a device for alcohol fermentation in this embodiment is the continuous-flow magnetic field-assisted fermentation and catalytic synthesis device in Embodiment 1, as shown in FIG. 3A and FIG. 3B. The continuous-flow magnetic field-assisted fermentation and catalytic synthesis device used in this embodiment is provided with three magnetic field assemblies 302, the magnetic field assemblies 302 are solenoid coils, where the wire diameter of the used solenoid coil is 2.6 mm, the number of turns of a single solenoid coil is 450 turns. The three magnetic field assemblies 302 are arranged in parallel and vertically fixed inside the heat-insulating container, respectively. The ratio of the outer perimeter of the solenoid coil to the center-to-center distance between the adjacent magnetic field assemblies is 1:2. The sample tube cavity 301 passes through the heat-insulating container and is made of a PFA material. A second constant-temperature cavity 303 is arranged around the sample tube cavity 301, and the solenoid coil is all wound around the periphery of the second constant-temperature cavity 303. The sample tubes 304 all pass through the sample tube cavities 301, and the fermentation liquid is pumped to flow continuously in the sample tubes 304, and an included angle 305 between the direction of the magnetic field line of the magnetic field and the flowing direction of the fermentation liquid in the sample tubes 304 is 15°. A constant-temperature water bath is pumped into the heat-insulating container, a refrigeration compressor or heat exchanger is configured to control the temperature of constant-temperature water bath. A peristaltic pump is configured to circulate the constant-temperature water bath through the first and second constant-temperature cavities of the heat-insulating container, as well as inside and outside the magnetic field assembly to ensure uniform thermal distribution.

A method for continuous-flow fermentation of alcohol under a magnetic field specifically includes:
(1) culture activation: a preserved strain of Saccharomyceses was inoculated onto the malt extract agar (MEA) medium and incubated at a constant temperature of 28° C. for two days to activate the strain;
(2) pretreatment of raw material: Saccharification liquid with the sugar degree of 12° Bx for industrial production of the alcohol was taken and sterilized, the activated Saccharomyceses strain was added in an inoculation amount of 3%, and uniformly mixed to obtain initial fermentation liquid; and
(3) the initial fermentation liquid in Step (2) was pumped into the continuous-flow magnetic field-assisted fermentation and catalytic device, device parameters were set, 7 A excitation current was applied to a solenoid coil within the magnetic field assembly to generate a static magnetic field with a magnetic field strength of 10 mT in the sample tube cavity 301. A fermentation temperature of the fermentation liquid was set to 28° C., a temperature of the constant-temperature water bath was controlled by a refrigeration compressor and heat exchanger, a peristaltic pump enabled the constant-temperature water bath to flow in and out of the heat-insulating container circularly, the temperature of the sample tube cavity 301 was thus kept at 28° C., and the fermentation liquid is subjected to the static magnetic field for continuous-flow constant-temperature culture at the flow rate of 4 L/h and at 28° C. for 5 days in the sample tube 304.

After that, the fermentation process of the alcohol was observed, and the alcohol content of the fermentation liquid was determined by an alcohol meter.

The result shows that in this embodiment, the alcohol content of the fermentation liquid is 9.74% alc/vol.

### Comparative Example 2

This comparative example provides a method for continuous-flow fermentation of alcohol under the magnetic field, a difference between Comparative Example 2 and Embodiment 2 is that the fermentation liquid is pumped into a continuous-flow magnetic field-assisted fermentation and catalytic device, an included angle between the direction of the magnetic field line of the magnetic field and the flowing direction of the fermentation liquid in the sample tubes is 105°, and the magnetic field strength produced by the device is 0.05 mT.

After that, referring to the method in Embodiment 2, the alcohol content of the fermentation liquid is determined. The result shows that the alcohol content of the fermentation liquid in Comparative Example 2 is 8.86% alc/vol.

By adopting the continuous-flow magnetic field-assisted fermentation and catalytic synthesis device provided by the present disclosure, compared with Comparative Example 2, the alcohol content of the fermentation liquid in Embodiment 2 is increased by 9.93%. Under the appropriate magnetic field strength as well as the included angle between the magnetic field direction and the flowing direction of the fermentation liquid, the interaction between the microorganisms and the substrate is enhanced, and water clusters are dispersed to promote alcohol fermentation process and its product yield. In Comparative Example 2, the magnetic field strength is extremely-low, and also the included angle between the flowing direction of the fermentation liquid and the magnetic field direction exceeds the threshold of 90°, the disturbance between different components is vigorous, making certain components in the fermentation liquid generate spin polarization or exposure of hydrophobic groups to a greater extent; and the interaction is reduced, there is no obvious non-thermal effect caused by the magnetic field, even side effect on the fermentation process, the energy utilization rate of the magnetic field and the alcohol yield after the fermentation are decreased.

### Embodiment 3

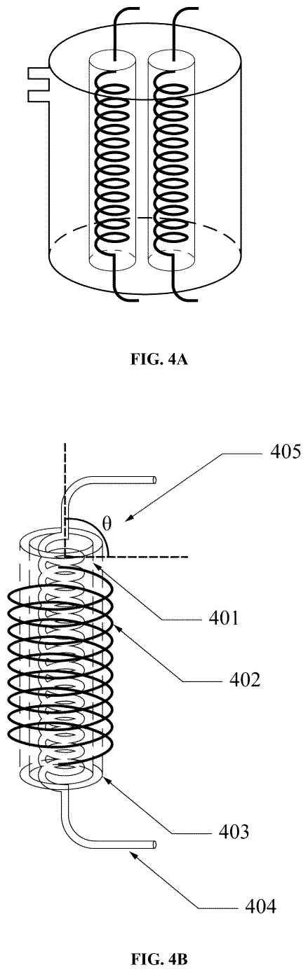

This embodiment provides a method for continuous-flow hydrolyzing polyferric sulfate under the magnetic field, a device for hydrolyzing polyferric sulfate in Embodiment 3 is the continuous-flow magnetic field-assisted fermentation and catalytic synthesis device in Embodiment 1, as shown in FIG. 4A and FIG. 4B. The continuous-flow magnetic field-assisted fermentation and catalytic synthesis device used in this embodiment is provided with two magnetic field assemblies 402, the magnetic field assemblies 402 are solenoid coils, where the wire diameter of the used solenoid coil is 2 mm, the number of turns of a single solenoid coil is 800 turns. The two magnetic field assemblies 402 are arranged in parallel and vertically fixed inside the heat-insulating container, respectively. A ratio of the outer perimeter of the solenoid coil to the center-to-center distance between the adjacent magnetic field assemblies is 1:1. The sample tube cavity 401 passes through the heat-insulating container and is made of an epoxy resin material, and a second constant-temperature cavity 403 is arranged around the sample tube cavity 401. The solenoid coils are all wound at the periphery of the second constant-temperature cavity 403. The sample tubes 404 all pass through the conducting cavity 401, and reaction solution is pumped into the sample tube 404 to flow continuously, an included angle 405 between the direction of the magnetic line of the magnetic field and the flowing direction of the reaction solution in the sample tube 404 is 90°. A constant-temperature oil bath is pumped into the heat-insulating container, the temperature of constant-temperature oil bath is controlled by a refrigeration compressor or heat exchanger. A centrifugal pump is configured to circulate the constant-temperature oil bath through the first and second constant-temperature cavities of the heat-insulating container, as well as inside and outside magnetic field to ensure uniform thermal distribution.

A method for continuous-flow hydrolyzing polyferric sulfate under the magnetic field specifically includes:
(1) polyferric sulfate liquid of 100 mg/L was prepared, where the content of ferric sulfate is 20% and the pH is 2.6; and
(2) the polyferric sulfate liquid in Step (1) was pumped into the continuous-flow magnetic field-assisted fermentation and catalytic synthesis device as a reaction solution, device parameters were set, 9.5 A excitation current was applied to a solenoid coil within the magnetic field assemblies to generate an alternating magnetic field with a magnetic field strength of 30 mT and a frequency of 800 Hz in the sample cavity 401, the hydrolysis temperature of the reaction solution was set to 65° C., a temperature of constant-temperature oil bath was controlled by a refrigeration compressor and a heat exchanger, a centrifugal pump enabled the constant-temperature oil bath to flow in and out of the heat-insulating container circularly, the temperature of the sample cavity 401 was thus kept at 65° C., and the reaction solution was subjected to the alternating magnetic field for continuous-flow constant-temperature hydrolysis at the flow rate of 10 L/h and at 65° C. for 2 hours in the sample tube 404.

After that, the hydrolysis process of the reaction solution was observed, the ultraviolet absorption spectrum scanning was carried out with a wavelength ranging from 200 to 400 nm, and the change of integral area of an absorption peak of Fe(OH)₂+ in the reaction solution was calculated through Gauss fitting. The integral area of the absorption peak indicates the content of Fe(OH)₂+ in the hydrolysis process. The result shows that after 2 hours of magnetic field-assisted hydrolysis, the integral area of the absorption peak of Fe(OH)₂+ in the reaction solution around 300 nm is 76.4.

### Comparative Example 3

This comparative example provides a method for continuous-flow hydrolysis of polyferric sulfate under the magnetic field, a difference between Comparative Example 3 and Embodiment 3 is that reaction solution is pumped into a continuous-flow magnetic field-assisted fermentation and catalytic device, an included angle between the direction of the magnetic field line of the magnetic field and the flowing direction of the reaction solution in the sample tube is 150°, and the device has the magnetic field with the strength of 600 mT and the frequency of 1500 Hz.

After that, the hydrolysis process of the reaction solution was observed, the ultraviolet absorption spectrum scanning was carried out with the wavelength ranging from 200 to 400 nm, and the change of integral area of an absorption peak of Fe(OH)₂+ in the reaction solution was calculated through Gauss fitting. The result shows that after 2 hours of magnetic field-assisted hydrolysis, the integral area of the absorption peak of Fe(OH)₂+ in the reaction solution around 300 nm is 48.2.

By using the continuous-flow magnetic field-assisted fermentation and catalytic synthesis device of the present disclosure, under the appropriate magnetic field strength and the included angle between the direction of the magnetic field line of the magnetic field and the flowing direction of the feed liquid passing through the sample tube, the reaction temperature of the sample is controllable. The compounds of metal salt have significant electron cloud orientation under the magnetic field. Charged solutes such as Fe³⁺ and free ions are subjected to Lorentz force, which enhancing the binding force of hydrated ionic compounds, improving the hydrogen bonding ability of water molecules, and increasing the mass transfer rate. Fe³⁺ is gradually bonded with hydroxy to promote the transformation from low polymeric state to high polymeric state hydrating compounds and thus promote hydrolysis reaction. In Comparative Example 3, the magnetic field strength is high, and the included angle between the flowing direction of the reaction solution and the magnetic field direction exceeds a threshold of 90°, making the disturbance between different components vigorously. Meanwhile, high spin polarization occurs in isocharged solutes and free ions in the polyferric sulfate solution, the interaction among these components is counteracted, then the growth of the high polymeric state substances is inhibited, the hydrolysis process is weakened. As a result, the integral area of the absorption peak around 300 nm in Comparative Example 3 is lowered when compared with Embodiment 3, that is, the content of Fe(OH)₂⁺ generated by hydrolysis is reduced by 36.9%.

It should be noted that the above is only some embodiments of the present disclosure. Referring to Embodiment 1, Embodiment 2 and Embodiment 3, the inventor changes at least one of the included angle θ between the direction of the magnetic field line of the magnetic field generated by the magnetic field assembly and the flowing direction of the feed liquid passing through the sample tube, the ratio of the outer perimeter of the magnetic field assembly to the center-to-center distance between the two adjacent magnetic field assemblies, the flow rate of the feed liquid in the sample tube, the temperatures of the magnetic field assembly and the constant-temperature medium, the type of the magnetic field, the magnetic field strength/frequency of the alternating magnetic field as well as the magnetic field strength of the static magnetic field. Specifically, the included angle θ between the direction of the magnetic field line of the magnetic field generated by the magnetic field assembly and the flowing direction of the feed liquid passing through the sample tube is set to 0°, 10°, 15°, 20°, 23°, 32°, 45°, 60°, 75°, 90°, etc., the temperatures of the magnetic field assembly and the constant-temperature medium are kept at −40° C., −30° C., −22° C., −10° C., 0° C., 15° C., 25° C., 55° C., 80° C., 100° C., 150° C., etc.; and the ratio of the outer perimeter of the magnetic field assembly to the center-to-center distance between the two adjacent magnetic field assemblies is set to 1:0.15, 1:0.3, 1:1.2, 1:2. 6, 1:4.3, 1:5.5, 1:6, 1:7.5, 1:9, 1:10, 1:15, 1:20, etc. The alternating magnetic field or static magnetic field is selected, the magnetic field strength of the alternating magnetic field is 0.1 mT, 1 mT, 10 mT, 50 mT, 100 mT, 260 mT, 350 mT, 400 mT, 420 mT, 500 mT, etc.; the magnetic field strength of the static magnetic field is 0.1 mT, 3 mT, 55 mT, 120 mT, 280 mT, 360 mT, 400 mT, 500 mT, 600 mT, etc. The flow rate of the feed liquid in the sample tube is 5 mL/h, 20 L/h, 100 L/h, 350 L/h, 420 L/h, 560 L/h, 600 L/h, 750 L/h, 900 L/h, 1000 L/h, etc. Therefore, series of embodiments are obtained, the influence of different magnetic field conditions on the fermentation of Antrodia camphorata, the production of the alcohol, the hydrolysis of the polyferric sulfate as well as the fermentation and synthesis reaction of other products are verified, and the results are basically close to those of Embodiment 1, Embodiment 2 and Embodiment 3.

It should be understood that the above embodiments are only used for describing the technical idea and features of the present disclosure, with the objective of enabling those skilled in the art to understand the contents of the present disclosure and to implement the present disclosure, but the scope of the present disclosure cannot be limited thereto. Equivalent changes or modifications made according to the spirit of the present disclosure should be covered within the protection scope of the present disclosure.

1. A continuous-flow magnetic field-assisted fermentation and catalytic synthesis device, comprising:
a constant-temperature unit, comprising a heat-insulating container, and a circulating constant-temperature medium supply system, wherein the heat-insulating container has a constant-temperature cavity and x sample tube cavities penetrating therethrough, the x sample tube cavities are encapsulated by the constant-temperature cavity and in heat-conducting fit with the constant-temperature cavity, and the circulating constant-temperature medium supply system is connected with the constant-temperature cavity to form a fluid loop for continuous-flow of a constant-temperature medium;
a reaction generation unit, comprising a sample container, and y sample tubes, wherein the sample container is at least configured to accommodate sample medium and provide an environment required for fermentation culture or catalytic synthesis reaction of the sample medium, each of the y sample tubes and the sample container are connected to form a fluid loop for the sample medium to flow continuously, and at least a part of each of the y sample tubes is arranged in one sample tube cavity and in heat-conducting fit with the sample tube cavity; and
a magnetic field generation unit, comprising y magnetic field assemblies, wherein the y magnetic field assemblies are arranged in the constant-temperature cavity, each of the y magnetic field assemblies is correspondingly distributed around one sample tube cavity, the magnetic field assembly is at least configured to generate a magnetic field inside the sample tube cavity, an included angle between a direction of a magnetic field line of a magnetic field generated by the magnetic field assembly and either an axial direction of the sample tube passing through each of the y magnetic field assemblies or a flowing direction of the sample medium within the sample tube ranges from 0° to 90°, the magnetic field is allowed to change physicochemical parameters of sample medium in the sample tube, x≥y≥1, and x and y are positive integers.

2. The continuous-flow magnetic field-assisted fermentation and catalytic synthesis device according to claim 1, wherein the constant-temperature unit further comprises y heat-dissipating structures, the y heat-dissipating structures are arranged in the constant-temperature cavity, and each of the y heat-dissipating structures is arranged around one sample tube cavity and in heat-conducting fit with the sample tube cavity.

3. The continuous-flow magnetic field-assisted fermentation and catalytic synthesis device according to claim 2, wherein the constant-temperature cavity comprises one first constant-temperature cavity, and y second constant-temperature cavities; the y second constant-temperature cavities are surrounded by and connected to the first constant-temperature cavity, each of the y second constant-temperature cavities is arranged around one sample tube cavity and in heat-conducting fit with the sample tube cavity, and the y second constant-temperature cavities and constant-temperature mediums filled in the y second constant-temperature cavities act as the y heat-dissipating structures; and/or
the heat-insulating container comprises a first housing and x second housings, the first housing is fixedly integrated with the x second housings, the first housing and the x second housings are enclosed to form the constant-temperature cavity, and each of the x second housings is enclosed to form the sample tube cavity, wherein the second housing is made of a nonmetallic material.

4. The continuous-flow magnetic field-assisted fermentation and catalytic synthesis device according to claim 2, wherein the magnetic field assembly is distributed in the heat-dissipating structure, or the magnetic field assembly is distributed around the sample tube cavity.

5. The continuous-flow magnetic field-assisted fermentation and catalytic synthesis device according to claim 2, wherein the magnetic field assembly comprises a metal coil, the metal coil is wound around a periphery of the sample tube cavity, and generates the magnetic field by electrically connected with a power supply; or the magnetic field assembly comprises at least one permanent magnet;
wherein a wire diameter of the metal coil ranges from 0.01 mm to 5 mm; and/or a number of turns of each metal coil ranges from 10 to 30000; and
wherein the magnetic field is an alternating magnetic field, or a static magnetic field; the alternating magnetic field is generated by introducing alternating current into the metal coil, and the static magnetic field is generated by introducing direct current into the metal coil; or the static magnetic field is generated by the at least one permanent magnet.

6. The continuous-flow magnetic field-assisted fermentation and catalytic synthesis device according to claim 5, wherein the y magnetic field assemblies are arranged in parallel, and a ratio of an outer perimeter of the magnetic field assembly to a center-to-center distance between the two adjacent magnetic field assemblies ranges from (1:0.15) to (1:20); and/or
a part, located in the sample tube cavity, of the sample tube is a straight tube, or spiral tube.

7. The continuous-flow magnetic field-assisted fermentation and catalytic synthesis device according to claim 1, wherein the constant-temperature unit comprises a temperature regulator, and a temperature sensor; the temperature regulator is connected to the heat-insulating container and the circulating constant-temperature medium supply system; the temperature regulator, the heat-insulating container and the circulating constant-temperature medium supply system form a fluid loop for continuous-flow of the constant-temperature medium; the temperature regulator is at least configured to maintain a temperature of the constant-temperature medium in the fluid loop, and the temperature sensor is at least configured to monitor a temperature of the constant-temperature medium flowing in and/out of the heat-insulating container.

8. A continuous-flow magnetic field-assisted fermentation and catalytic synthesis method, comprising:
providing the continuous-flow magnetic field-assisted fermentation and catalytic synthesis device according to claim 1;
enabling the sample medium in the sample container to pass through the sample tube in continuous-flow state;
enabling the magnetic field assembly to generate the magnetic field passing through the sample tube, wherein the included angle between the direction of the magnetic field line of the magnetic field generated by the magnetic field assembly and either the axial direction of the sample tube passing through each of the y magnetic field assemblies or the flowing direction of the sample medium in the sample tube ranges from 0° to 90°; and
enabling the constant-temperature unit to maintain temperatures of the magnetic field assembly and the sample medium in the sample tube.

9. The continuous-flow magnetic field-assisted fermentation and catalytic synthesis method according to claim 8, wherein the magnetic field is an alternating magnetic field, or a static magnetic field, the alternating magnetic field has magnetic field strength ranging from 0.1 mT to 500 mT and a frequency ranging from 0.1 Hz to 100 kHz, and the static magnetic field has a magnetic field strength ranging from 0.1 mT to 600 mT.

10. The continuous-flow magnetic field-assisted fermentation and catalytic synthesis method according to claim 8, wherein a flow rate of the sample medium in the sample tube ranges from 5 mL/h to 1000 L/h.

11. The continuous-flow magnetic field-assisted fermentation and catalytic synthesis device according to claim 3, wherein the magnetic field assembly is distributed in the heat-dissipating structure, or the magnetic field assembly is distributed around the sample tube cavity.

12. The continuous-flow magnetic field-assisted fermentation and catalytic synthesis device according to claim 3, wherein the magnetic field assembly comprises a metal coil, the metal coil is wound around a periphery of the sample tube cavity, and generates the magnetic field by electrically connected with a power supply; or the magnetic field assembly comprises at least one permanent magnet;
wherein a wire diameter of the metal coil ranges from 0.01 mm to 5 mm; and/or a number of turns of each metal coil ranges from 10 to 30000; and
wherein the magnetic field is an alternating magnetic field, or a static magnetic field; the alternating magnetic field is generated by introducing alternating current into the metal coil, and the static magnetic field is generated by introducing direct current into the metal coil; or the static magnetic field is generated by the at least one permanent magnet.

13. The continuous-flow magnetic field-assisted fermentation and catalytic synthesis device according to claim 12, wherein the y magnetic field assemblies are arranged in parallel, and a ratio of an outer perimeter of the magnetic field assembly to a center-to-center distance between the two adjacent magnetic field assemblies ranges from (1:0.15) to (1:20); and/or
a part, located in the sample tube cavity, of the sample tube is a straight tube, or spiral tube.

14. The continuous-flow magnetic field-assisted fermentation and catalytic synthesis method according to claim 8, wherein in the continuous-flow magnetic field-assisted fermentation and catalytic synthesis device, the constant-temperature unit further comprises y heat-dissipating structures, the y heat-dissipating structures are arranged in the constant-temperature cavity, and each of the y heat-dissipating structures is arranged around one sample tube cavity and in heat-conducting fit with the sample tube cavity.

15. The continuous-flow magnetic field-assisted fermentation and catalytic synthesis method according to claim 14, wherein in the continuous-flow magnetic field-assisted fermentation and catalytic synthesis device, the constant-temperature cavity comprises one first constant-temperature cavity, and y second constant-temperature cavities; the y second constant-temperature cavities are surrounded by and connected to the first constant-temperature cavity, each of the y second constant-temperature cavities is arranged around one sample tube cavity and in heat-conducting fit with the sample tube cavity, and the y second constant-temperature cavities and constant-temperature mediums filled in the y second constant-temperature cavities act as the y heat-dissipating structures; and/or
the heat-insulating container comprises a first housing and x second housings, the first housing is fixedly integrated with the x second housings, the first housing and the x second housings are enclosed to form the constant-temperature cavity, and each of the x second housings is enclosed to form the sample tube cavity, wherein the second housing is made of a nonmetallic material.

16. The continuous-flow magnetic field-assisted fermentation and catalytic synthesis method according to claim 14, wherein in the continuous-flow magnetic field-assisted fermentation and catalytic synthesis device, the magnetic field assembly is distributed in the heat-dissipating structure, or the magnetic field assembly is distributed around the sample tube cavity.

17. The continuous-flow magnetic field-assisted fermentation and catalytic synthesis method according to claim 14, wherein in the continuous-flow magnetic field-assisted fermentation and catalytic synthesis device, the magnetic field assembly comprises a metal coil, the metal coil is wound around a periphery of the sample tube cavity, and generates the magnetic field by electrically connected with a power supply; or the magnetic field assembly comprises at least one permanent magnet;
wherein a wire diameter of the metal coil ranges from 0.01 mm to 5 mm; and/or a number of turns of each metal coil ranges from 10 to 30000; and
wherein the magnetic field is an alternating magnetic field, or a static magnetic field; the alternating magnetic field is generated by introducing alternating current into the metal coil, and the static magnetic field is generated by introducing direct current into the metal coil; or the static magnetic field is generated by the at least one permanent magnet.

18. The continuous-flow magnetic field-assisted fermentation and catalytic synthesis method according to claim 17, wherein in the continuous-flow magnetic field-assisted fermentation and catalytic synthesis device, the y magnetic field assemblies are arranged in parallel, and a ratio of an outer perimeter of the magnetic field assembly to a center-to-center distance between the two adjacent magnetic field assemblies ranges from (1:0.15) to (1:20); and/or
a part, located in the sample tube cavity, of the sample tube is a straight tube, or spiral tube.

19. The continuous-flow magnetic field-assisted fermentation and catalytic synthesis method according to claim 8, wherein in the continuous-flow magnetic field-assisted fermentation and catalytic synthesis device, the constant-temperature unit comprises a temperature regulator, and a temperature sensor; the temperature regulator is connected to the heat-insulating container and the circulating constant-temperature medium supply system; the temperature regulator, the heat-insulating container and the circulating constant-temperature medium supply system form a fluid loop for continuous-flow of the constant-temperature medium; the temperature regulator is at least configured to maintain a temperature of the constant-temperature medium in the fluid loop, and the temperature sensor is at least configured to monitor a temperature of the constant-temperature medium flowing in and/out of the heat-insulating container.

20. The continuous-flow magnetic field-assisted fermentation and catalytic synthesis method according to claim 14, wherein the magnetic field is an alternating magnetic field, or a static magnetic field, the alternating magnetic field has magnetic field strength ranging from 0.1 mT to 500 mT and a frequency ranging from 0.1 Hz to 100 kHz, and the static magnetic field has a magnetic field strength ranging from 0.1 mT to 600 mT.
